(19) United States
(12) Patent Application Publication
(10) Pub. No.: US 2014/0005131 A1
(43) Pub. Date: Jan. 2, 2014

(54) ACTIVE INGREDIENT COMBINATIONS OF GLUCOSYL GLYCERIDES AND ONE OR MORE PRESERVATIVES

(71) Applicants: Rainer Kroepke, Schenefeld (DE); Christian Frese, Hamburg (DE); Cathrin Scherner, Norderstedt (DE); Martin Griebenow, Hamburg (DE); Svenja Lena Moellgaard, Hamburg (DE)

(72) Inventors: Rainer Kroepke, Schenefeld (DE); Christian Frese, Hamburg (DE); Cathrin Scherner, Norderstedt (DE); Martin Griebenow, Hamburg (DE); Svenja Lena Moellgaard, Hamburg (DE)

(73) Assignee: BEIERSDORF AG, Hamburg (DE)

(21) Appl. No.: 13/996,630

(22) PCT Filed: Nov. 28, 2011
(86) PCT No.: PCT/EP2011/071138

§ 371 Date: Sep. 16, 2013

(30) Foreign Application Priority Data
Dec. 23, 2010 (DE) 10 2010 055 768.4

(51) Int. Cl. A61K 31/7032; A61K 8/60; A61K 8/44; A61Q 19/00; A61K 8/49; A61K 8/41

(52) U.S. Cl. CPC A61K 31/7032; A61K 8/49; A61K 8/416; A61K 8/4926; A61K 8/442; A61Q 19/007; A61K 8/602
USPC 514/25

(57) ABSTRACT

Ingredient combinations of (i) one or more preservatives selected from the group of the isothiazolinones, benzethonium chloride, piroctone olamine, and ethyl lauroyl arginate, and (ii) one or more glucosyl glycerides.

The present invention relates to active ingredient combinations of glucosyl glycerides and one or more preservatives and the use thereof in the field of cosmetic and pharmaceutical dermatology.

In particular, the present invention relates to active ingredients and cosmetic or dermatological preparations comprising such active ingredient combinations.

The outermost layer of the epidermis, the stratum corneum (horny layer), is an important barrier layer of particular importance for, inter alia, protection against environmental influences and from drying out. The horny layer is continuously worn away in contact with the environment and must therefore be continuously replaced.

A skin model widely used in professional circles today regards the stratum corneum as a two-component system similar to a brick wall (brick and mortar model). In this model, the corneocytes (horny cells) correspond to the bricks, while the complex composite lipid membrane in the intercellular spaces corresponds to the mortar.

Apart from their barrier effect against external chemical and physical influences, the epidermal lipids also contribute to the cohesion of the horny layer and affect the skin smoothness. In contrast to the sebaceous gland lipids which do not form a closed film on the skin, the epidermal lipids are distributed over the whole horny layer.

The extremely complex interaction of the moisture-binding substances and the lipids of the upper skin layers is very important for the regulation of skin moisture. Therefore, cosmetics generally comprise water-binding substances in addition to balanced lipid mixtures and water. These include, inter alia, polyols such as glycerol, sorbitol and xylitol, ethoxylated polyols and hydrolysed proteins. Substances containing so-called natural moisturizing factor (NMF) are further used, such as urea, carbohydrates (e.g. glucose) and amino acids (e.g. serine). These substances are therefore particularly important for the care performance of a cosmetic product, especially due to their relatively good skin and mucous membrane compatibility. The desire for clean skin is probably as old as humankind, since dirt, sweat and residual dead skin particles offer the ideal breeding ground for pathogens and parasites of every type. The desire for personal hygiene steadily increased since in the 60s of the 20^{th} century, in addition to “classical” soap, liquid detergents with newly developed synthetic surfactants were formulated. Since then, bathing and showering are now indispensable in our daily life. Nowadays, a variety of products for the cleaning of various parts of the body are available to consumers.

Cleaning means the removal of (environmental) dirt and this causes an increase in physical and mental well-being. The cleaning of the surface of skin and hair is a very complex process dependent on many parameters. Firstly, external substances, such as hydrocarbons or inorganic pigments from different environments and also residues of cosmetics or even undesired microorganisms should be removed as completely as possible. Secondly, endogenous secretions such as sweat, sebum, skin flakes and hair dandruff should be washed off without profound changes to the physiological equilibrium.

The preserving of cosmetic preparations is a difficult task, since, on the one hand, a sufficient preservation to protect the formulation from bacterial contamination must be ensured while, on the other hand, negative influences of the preservatives on the compatibility, stability and organoleptic properties of the formulation must be avoided. The preservatives must therefore be effective, toxicologically harmless, well tolerated by skin, be stable to formulation and inexpensive to produce.

A variety of preservatives, such as formaldehyde eliminators, parabens, phenols and derivatives thereof, bisguanidines and halogenated compounds, have a high antimicrobial efficacy, but can however cause skin irritation and allergic reactions and are therefore regarded critically by consumers and mostly regarded as undesirable.

Paraben-free preservatives, such as isothiazolinones, benzethonium chloride, piroctone olamine and lauroyl ethyl arginate are used as preservative. What they have in common is that they produce a slippery skin feel on the skin when diluted with water and have unsatisfactory viscosity characteristics at very low temperatures. Under these conditions, such preparations can no longer be removed from the packaging.

Thus, there is a need for preparations which overcome the disadvantages of the prior art.

Following this, it was surprising and unpredictable that active ingredient combinations of
(i) one or more preservatives selected from the group of isothiazolinones, benzethonium chloride, piroctone olamine and lauroyl ethyl arginate and
(ii) one or more glucosyl glycerides
   or cosmetic preparations containing such active ingredient combinations eliminate the disadvantages of the prior art.

The object was surprisingly solved by the use of glyceryl glucosides, particularly in aqueous cleaning formulations comprising specific preservatives. These preparations are characterized by excellent organoleptic properties & an improved viscosity ratio at low and high temperatures.

Of advantage in the scope of the present invention glucosyl glycerides of the general formula

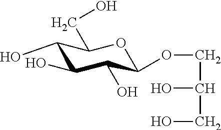

and/or of the general formula

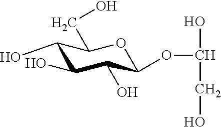

and/or of the general formula

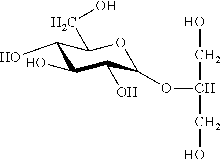

and/or of the general formula

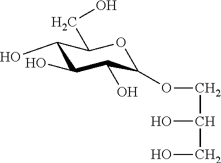

are preferred in accordance with the invention.

Particularly advantageous according to the invention are those preparations which are characterized in that the glucosyl glyceride(s) are present in the water and/or oil phase at concentrations of 0.001-40.00% by weight, preferably 0.005-15.00% by weight, particularly preferably 0.01-12.00% by weight, based in each case on the total weight of the composition.

The preferred concentration of one or more preservatives, selected from the group of the isothiazolinones, benzethonium chloride, piroctone olamine and lauroyl ethyl arginate, is 0.00001 to 10% by weight, particularly preferably 0.001-5% by weight, particularly preferably 0.005%-3% by weight, based on the total weight of the preparations.

The use of the preparation according to the invention as a cosmetic cleaning preparation is advantageous in accordance with the invention. The preparation according to the invention is preferably used as a shower gel, foam bath and bath soak, shampoo and/or facial cleanser.

Cosmetic and dermatological preparations according to the invention may exist in different forms. Thus, they may be e.g. a solution, an anhydrous preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or else an aerosol.

It is advantageous according to the invention if the inventive cosmetic preparation is stored in a bottle, squeeze bottle, pump spray or aerosol can and is dispensed therefrom. Accordingly, bottles, squeeze bottles, double-chamber packing means, pump spray cans or aerosol cans which contain a preparation according to the invention are in accordance with the invention.

The preparation according to the invention advantageously contains one or more anionic surfactants. According to the invention, these are advantageously present in the preparation at a concentration of 1 to 20% by weight, and according to the invention preferably at a concentration of 5 to 12% by weight, in each case based on the total weight of the preparation. According to the invention particular preference is given here to the use of sodium lauryl ether sulfate as anionic surfactant.

According to the invention, the preparation according to the invention advantageously contains one or more amphoteric surfactants. According to the invention, these are advantageously present in the preparation at a concentration of 1 to 20% by weight, and according to the invention preferably at a concentration of 3 to 8% by weight, in each case based on the total weight of the preparation. According to the invention particular preference is given here to the use of cocamidopropyl betaine as amphoteric surfactant.

Moreover, the preparation according to the invention may advantageously also contain non-ionic surfactants. Particular preference is given here, according to the invention, to the use of PEG-7 glyceryl cocoate and/or PEG-40 hydrogenated castor oil as non-ionic surfactant.

It is likewise advantageous, within the context of the present invention, to add cationic polymers to the preparations. Suitable cationic polymers are, for example,
quaternized cellulose derivatives, such as polyquaternium-10, as are commercially available under the names Celquat and Polymer JR
cationic guar derivatives, such as in particular the products sold under the trade names Cosmedia Guar and Jaguar
polymeric dimethyldiallylammonium salts and copolymers thereof with esters and amides of acrylic acid and methacrylic acid, such as in particular the products commercially available under the names Merquat 100 and Merquat 550.

Within the context of the present invention, it is especially advantageous to use cationic polymer or mixtures of cationic polymers at a concentration of 0.01 to 2% by weight, preferably at a concentration of 0.05 to 1.5% by weight and particularly preferably from 0.1 to 1.0% by weight, in each case based on the total weight of the preparation.

Apart from the aforementioned substances, the compositions according to the invention optionally contain the additives customary in cosmetics, for example, perfume, dyes, antimicrobial substances, refatting agents, complexing and sequestering agents, pearlescent agents, plant extracts, vitamins, active ingredients, preservatives, bactericides, pigments which have a coloring effect, thickeners, softening, moisturizing and/or humectant substances, or other customary constituents of a cosmetic or dermatological formulation such as alcohols, polyols, polymers, foam stabilizers, electrolytes, organic solvents or silicone derivatives.

The active ingredients, auxiliaries and additives which can be used advantageously according to the invention are here in no way limited to the substances and compounds mentioned here by name.

As conventional cosmetic carrier substances for the preparation of the deodorizing preparations according to the use according to the invention, skin care lipids or lipid like substances, such as decyl oleate, cetyl alcohol, cetylstearyl alcohol and 2-octyldodecanol, may be used for such preparations in the customary amount ratios therefor, in addition to water, ethanol and isopropanol, glycerol and propylene glycol, and also mucus-forming substances and thickeners, e.g. hydroxyethyl- or hydroxypropylcellulose, polyacrylic acid, polyvinylpyrrolidone, and also cyclic silicone oils (polydimethylsiloxanes) in small amounts and liquid polymethylphenylsiloxanes of low viscosity.

It is likewise advantageous to add customary antioxidants to the preparations within the context of the present invention. According to the invention, favorable antioxidants which may be used are all antioxidants that are suitable or customary for cosmetic and/or dermatological applications.

The amount of antioxidants (one or more compounds) in the preparations is preferably 0.001 to 30% by weight, particularly preferably 0.05-20% by weight, in particular 1-10% by weight, based on the total weight of the preparation.

If the cosmetic or dermatological preparation within the context of the present invention is a solution or emulsion or dispersion, solvents which can be used are:
water or aqueous solutions
oils, such as triglycerides of capric acid or of caprylic acid, but preferably castor oil;
fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids;
alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the aforementioned solvents are used. In the case of alcoholic solvents, water may be a further constituent.

If the cosmetic or dermatological preparation within the context of the present invention is a solution or emulsion or dispersion, solvents which can be used are:
water or aqueous solutions
oils, such as triglycerides of capric acid or of caprylic acid, but preferably castor oil;
fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids;
alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In alcoholic solvents, water can be a further constituent.

For the purposes of the present invention, suitable propellants for cosmetic and/or dermatological preparations which can be sprayed from aerosol containers are the customary known readily volatile, liquefied propellants, for example hydrocarbons (propane, butane, isobutane), which can be used alone or in a mixture with one another. Compressed air is also used advantageously.

The person skilled in the art is of course aware that there are propellant gases which are non-toxic per se which would be suitable in principle for realizing the present invention in the form of aerosol preparations, but which must nevertheless be avoided because of their harmful impact on the environment or other accompanying circumstances, in particular fluorinated hydrocarbons and chlorofluorocarbons (CFCs).

Cosmetic preparations within the context of the present invention can also be present as gels which comprise, besides an effective content of the active ingredient according to the invention and solvents customarily used therefor, preferably water, also organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates such as bentonites, or a mixture of polyethylene glycol and polyethylene glycol stearate or distearate. The thickener is present in the gel e.g. in an amount between 0.1 and 30% by weight, preferably between 0.5 and 15% by weight.

Advantageous embodiments of the present invention follow.

### Methods:

The measurements were carried out under the following conditions:
Instrument: Ares 8
Measurement: GEFR (rheological freezing point determination)
Program: dynamic temperature steps from 25° C. to −30° C. in 1° C. increments
Measuring system: plate/plate 25 mm, 1 mm gap

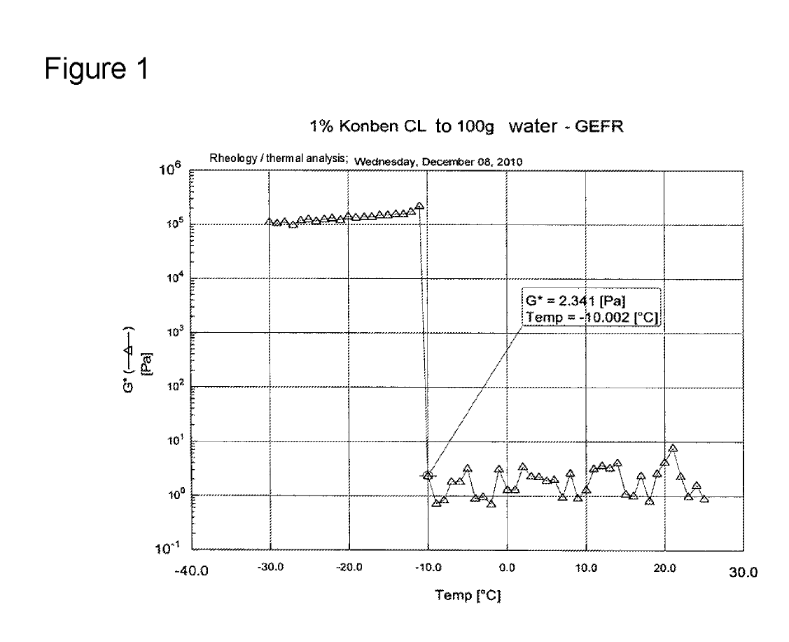

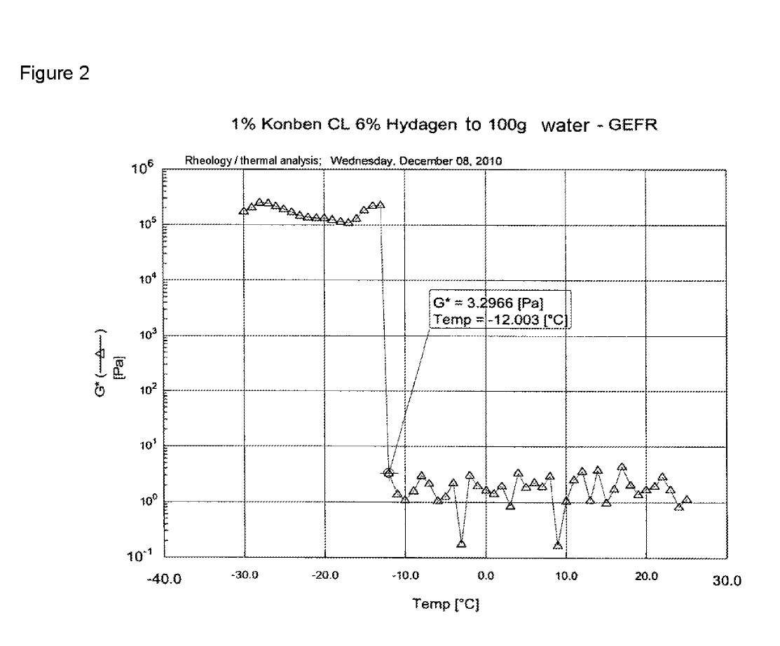

### See FIGS. 1 and 2

### Example Formulations

### Shampoo Preparations

all concentration data in % by weight

| Phase | INCI | 1.1 | 1.2 | 1.3 | 1.4 |
|---|---|---|---|---|---|
| A | Water, demin. | to 100 | to 100 | to 100 | to 100 |
| A | Texapon N 70 | 10.50 | 12.00 | 6.50 | 13.00 |
| A | Rewopol SB CS 50 | — | 7.25 | — | — |
| A | Tego Betain F 50 | 11.50 | 9.50 | 13.00 | 10.00 |
| A | Merquat 550 | 2.50 | — | 2.50 | 2.50 |
| A | Panthenol | — | 0.50 | 1.00 | 0.10 |
| A | Methylisothiazolinone | 0.10 | 0.05 | 0.01 | 0.20 |
| A | Benzethonium chloride | 0.01 | 0.10 | 0.03 | 0.20 |
| A | Glyceryl glucoside | 0.05 | 0.50 | 1.00 | 5.00 |
| B | Ucare Polymer JR 400 | 0.10 | 0.30 | 0.10 | — |
| B | Water, demin. | 15.00 | 15.00 | 15.00 | — |
| C | Water, demin. | — | 10.00 | 10.00 | — |
| C | Sodium hydroxide soln. | — | 0.01 | 0.01 | — |
| | 45% | | | | |
| C | Jaguar Excel | — | 0.10 | 0.12 | — |
| C | Citric acid | — | 0.03 | 0.03 | — |
| D | Eumulgin HRE 40 | 0.30 | 0.40 | 0.30 | 0.40 |
| D | Jojoba oil | 0.01 | 0.01 | 0.01 | 0.01 |
| D | Perfume | 0.60 | 0.80 | 0.90 | 0.70 |
| E₁ | Euperlan PK 900 | 4.00 | 6.00 | — | — |
| E₂ | Sodium chloride* | 2.20 | 1.80 | 1.80 | 1.30 |
| E₃ | Citric acid** | 0.15 | 0.10 | 0.40 | 0.22 |

*amount variable to establish a viscosity in the range of 3000-4500 mPas (measured using a HAAKE viscotester VT02 with rotor 1)
**amount variable to establish a pH of 4.8 to 5.8

The ingredients of phase A are mixed with one another until a homogeneous phase is formed.

Ucare Polymer JR 400 is dispersed in the water of phase B. Phase B is heated to ca. 70° C. with stirring until a clear solution is formed. Phase B is cooled and added to phase A.

The water of phase C is mixed with the sodium hydroxide solution. Jaguar Excel is slowly interspersed with stirring. The mixture is then heated to 70° C. and stirred until a homogeneous phase has been formed. The citric acid is added with stirring. Phase C is cooled and added to phase A.

Eumulgin HRE 40 is melted at a temperature of ca. 40° C. The other raw materials of phase D are added to the Eumulgin HRE 40. The phase is homogeneously mixed and added to phase A.

The addition of the ingredients of phase E is carried out in the order stated. It is stirred until a homogeneous shampoo is formed.

### Shower Gel Preparations

all concentration data in % by weight

| Phase | INCI | 2.1 | 2.2 | 2.3 | 2.4 | 2.5 |
|---|---|---|---|---|---|---|
| A | Water, demin. | to 100 | to 100 | to 100 | to 100 | to 100 |
| A | Texapon N70 | 9.00 | 11.00 | 7.00 | 4.00 | 6.50 |
| A | Piroctone olamine | 0.10 | 0.05 | 0.20 | 0.20 | 0.01 |
| A | Benzethonium | 0.20 | — | 0.02 | — | 0.10 |
| | chloride | | | | | |
| A | Citric acid* | 0.35 | 0.35 | 0.35 | 0.35 | 0.50 |
| A | Rewoderm | 0.10 | 0.10 | 0.10 | 0.10 | |
| | LI 520-70** | | | | | |
| A | Glucamate | — | — | — | — | 0.03 |
| | DOE-120 | | | | | |
| A | Glycerol | — | — | 1.00 | — | 1.00 |
| A | Uvinul MS-40 | — | — | — | 0.50 | — |
| A | Glyceryl | 0.01 | 0.10 | 3.00 | 1.00 | 1.00 |
| | glucoside | | | | | |
| B | Eumulgin | 0.50 | 0.50 | 0.50 | 0.50 | 0.25 |
| | HRE 40 | | | | | |
| B | Almond oil | 0.01 | 0.01 | — | — | 0.20 |
| B | Sunflower oil | — | — | 0.01 | 0.04 | — |
| B | Avocado oil | — | — | — | 0.02 | — |
| B | Cetiol HE | 1.75 | 2.00 | 2.50 | 2.00 | 1.00 |
| B | Polyox WSR-301 | — | — | — | 0.10 | — |
| B | Perfume | 1.00 | 1.00 | 1.00 | 1.00 | 0.20 |
| C₁ | Merquat 550 | 3.00 | 6.00 | 4.00 | 5.00 | — |
| C₂ | Tego Betain F 50 | 15.00 | 20.00 | 12.00 | 25.00 | 15.00 |
| C₃ | Opulyn 301 | 1.00 | 1.00 | 1.00 | — | — |
| C₄ | Euperlan PK 900 | — | — | — | 4.00 | — |
| C₅ | Euperlan PK 771 | — | — | — | — | 4.00 |

*amount variable to establish a pH of 4.8 to 5.3
**amount variable to establish a viscosity in the range of 3000-5000 mPas (measured using a HAAKE viscotester VT02 with rotor 1)

The ingredients of phase A are mixed with one another until a homogeneous mixture is formed.

Eumulgin HRE 40 is melted at a temperature of ca. 40° C. The other raw materials of phase B are added to the Eumulgin HRE 40. The phase is homogeneously mixed and added to phase A.

The addition of the ingredients of phase C is carried out in the order stated, with stirring.

### Rinse-Off Conditioner

all concentration data in % by weight

| Phase | INCI | 3.1 | 3.2 |
|---|---|---|---|
| A | Water, demin. | to 100 | to 100 |
| A | Dehyton AB 30 | 1.00 | 1.00 |
| A | Ethyl lauroyl arginate | 0.10 | 0.25 |
| A | Lactic acid* | 0.60 | 0.40 |
| A | Dehyquart A-CA | 4.00 | 2.00 |
| A | Sodium chloride | 0.07 | 0.07 |
| A | Glyceryl glucoside | 0.10 | 1.00 |
| B | Water, demin. | 10.00 | 10.00 |
| B | Sodium hydroxide soln. | 0.02 | 0.02 |
| | 45% | | |
| B | Jaguar Excel | 0.10 | 0.10 |
| B | Citric acid | 0.04 | 0.04 |
| C | Lanette O | 5.00 | 5.20 |
| C | Tego Amid S 18 | 1.50 | 1.00 |
| D | DC 5-7139 | 4.60 | 1.50 |
| E | Perfume | 0.80 | 0.70 |

*amount variable to establish a pH of the water phase of 3.8 to 4.8

The raw materials of phase A are mixed with one another and heated to 80° C.

The water of phase B is mixed with the sodium hydroxide solution. Jaguar Excel is slowly interspersed with stirring. The mixture is then heated to 70° C. and stirred until a homogeneous phase has been formed. The citric acid is added with stirring. Phase B is added to phase A.

The raw materials of phase C are heated to 80° C. in a Kitchen Aid and homogeneously mixed with stirring. Phase A is added to phase C.

Phase A and phase C are mixed with one another in a Kitchen Aid and cooled. Phase C is added at 40° C. and phase D at 30° C. It is stirred until a homogeneous conditioner is formed.

The viscosity of the conditioners is 3000-4000 mPas (measured using a HAAKE viscotester VT02 with rotor 1).

### W/O Emulsions

| | 1 | 2 | 3 | 4 | 5 |
|---|---|---|---|---|---|
| Triglycerol diisostearate | 1.0 | 0.5 | 0.25 | 2.0 | 3.0 |
| Diglycerol | 1.0 | 1.5 | 1.75 | 3.0 | 2.0 |
| dipolyhydroxystearate | | | | | |
| Paraffin oil | 12.5 | 10.0 | 8.0 | 5.0 | 11.5 |
| Vaseline | 8.0 | 6.0 | 5.0 | 12.0 | 2.5 |
| Hydrogenated coco | 2.0 | 1.0 | 2.5 | 5.0 | 0.25 |
| glycerides | | | | | |
| Decyl oleate | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Glyceryl glucoside | 7.5 | 12.5 | 2.5 | 5 | 20 |
| Magnesium sulfate | 0.5 | 0.6 | 0.5 | 0.7 | 1.0 |
| Glycerol | 3.0 | 5.0 | 10.0 | 15.0 | 1.5 |
| Perfume | qs | qs | qs | qs | qs |
| Ethanol | 2.0 | — | 5.0 | — | — |
| Caprylic/capric acid | 2.0 | 2.5 | 3.0 | 5.0 | 0.5 |
| triglyceride | | | | | |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Water | to 100 | to 100 | to 100 | to 100 | to 100 |

### W/O Emulsions

| | 6 | 7 | 8 | 9 | 10 |
|---|---|---|---|---|---|
| PEG-30 | — | 0.5 | 0.25 | — | 3.0 |
| dipolyhydroxystearate | | | | | |
| Lanolin alcohol | 1.0 | 1.5 | 1.75 | 3.0 | — |
| Paraffin oil | 12.5 | 10.0 | 8.0 | 5.0 | 10.5 |
| Vaseline | 8.0 | 6.0 | 5.0 | 12.0 | 2.5 |
| Hydrogenated coco | 2.0 | 1.0 | 2.5 | 5.0 | 0.25 |
| glycerides | | | | | |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Aluminium stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Microcrystalline | 0.5 | 1.0 | 0.75 | 0.25 | 0.1 |
| cellulose | | | | | |
| Magnesium sulfate | 0.5 | 0.6 | 0.5 | 0.7 | 1.0 |
| Glycerol | 3.0 | 5.0 | 10.0 | 15.0 | 1.5 |
| Citric acid | 0.2 | 0.1 | 0.2 | 0.3 | 1.0 |
| Perfume | qs | qs | qs | qs | qs |
| 1,3-Butylene glycol | 2.0 | — | 5.0 | — | — |
| Glyceryl glucoside | 2.0 | 2.5 | 3.0 | 5.0 | 0.5 |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Talc | — | — | 0.05 | — | 0.1 |
| Water | to 100 | to 100 | to 100 | to 100 | to 100 |

### W/S Emulsion

| | 11 | 12 | 13 | 14 | 15 |
|---|---|---|---|---|---|
| Cetyl PEG/PPG-10/1 | 1.0 | — | — | 3.0 | 5.0 |
| dimethicone | | | | | |
| Cyclomethicone + | 10.0 | 12.5 | 25 | — | — |
| PEG/PPG-18/18 | | | | | |
| dimethicone (90:10) | | | | | |
| Cyclic silicone oil | 12.5 | 15 | 22.0 | 20.0 | 15.5 |
| (cycIomethicone) | | | | | |
| Linear silicone oil | 5.0 | 13.0 | 5.0 | 12.0 | 15.0 |
| (dimethicone) | | | | | |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glycerol | 3.0 | 5.0 | 10.0 | 15.0 | 1.5 |
| Citric acid | 0.2 | 0.1 | 0.2 | 0.3 | 1.0 |
| Sodium citrate | 1.0 | 0.1 | 0.4 | 0.9 | 2.5 |
| Perfume | qs | qs | qs | qs | qs |
| Glyceryl glucoside | 10.0 | 15.0 | 7.5 | 5.0 | 2.5 |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Modified starch | — | 2.5 | — | 0.15 | — |
| Water | to 100 | to 100 | to 100 | to 100 | to 100 |

### W/S Emulsions

| | 16 | 17 | 18 | 19 | 20 |
|---|---|---|---|---|---|
| Cetyl PEG/PPG-10/1 | 1.0 | — | — | 3.0 | 5.0 |
| dimethicone | | | | | |
| Cyclomethicone + | 10.0 | 12.5 | 25 | — | — |
| PEG/PPG-18/18 | | | | | |
| dimethicone (90:10) | | | | | |
| Cyclic silicone oil | 12.5 | 15 | 8.0 | 20.0 | 17.5 |
| (cycIomethicone) | | | | | |
| Linear silicone oil | 5.0 | 13.0 | 5.0 | 12.0 | 15.0 |
| (dimethicone) | | | | | |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glyceryl glucoside | 3.0 | 5.0 | 10.0 | 15.0 | 1.5 |
| Lactic acid | 0.2 | 0.1 | 0.2 | — | — |
| Sodium lactate | 0.2 | 1.0 | 0.05 | — | — |
| Perfume | qs | qs | qs | qs | qs |
| Glycerol | 1.0 | 0.1 | 1.5 | 2.5 | 0.1 |
| Stearyldimethicone | 0.5 | — | 0.7 | — | — |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Modified starch | — | 2.5 | — | 0.15 | — |
| Water | to 100 | to 100 | to 100 | to 100 | to 100 |

### Silicone-in-Water Emulsion

| | 21 | 22 | 23 | 24 | 25 |
|---|---|---|---|---|---|
| Bis-PEG/PPG-16/16 | 1.0 | 2.0 | 8.0 | 3.0 | 5.0 |
| PEG/PPG, | | | | | |
| 16/16 dimethicone | | | | | |
| caprylic/capric | | | | | |
| triglyceride | | | | | |
| Cyclic silicone oil | 12.5 | 15 | 25.0 | 10.0 | 7.5 |
| (cycIomethicone) | | | | | |
| Linear silicone oil | 5.0 | 15.0 | 5.0 | 12.0 | 15.0 |
| (dimethicone) | | | | | |
| Octyldodecanol | 0.5 | 1.0 | 0.75 | 3.0 | 0.25 |
| Glycerol | 5.0 | 7.5 | 10.0 | 3.0 | 1.0 |
| Panthenol | 0.5 | 1.0 | 0.75 | 0.25 | 0.1 |
| Perfume | qs | qs | qs | qs | qs |
| Stearyl dimethicone | 0.5 | — | 0.7 | — | — |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Glyceryl glucoside | 5.0 | 2.5 | 7.5 | 15.0 | 12.5 |
| Water | to 100 | to 100 | to 100 | to 100 | to 100 |

### O/W Emulsion

| | 26 | 27 | 28 | 29 | 30 |
|---|---|---|---|---|---|
| Glyceryl stearate citrate | 1.0 | 1.5 | 1.5 | 0.5 | 0.25 |
| Cetyl stearyl alcohol | 2.5 | 5 | 0.5 | 2.0 | 1.5 |
| Ethylhexyl | 5.0 | 3.0 | 2.0 | 2.0 | 5.0 |
| methoxycinnamate | | | | | |
| EthyIhexyIgIycerin | 1 | — | 0.5 | 1 | — |
| Aluminum chlorohydrate | — | 1 | 0.02 | 1 | — |
| Ethanol | — | — | 3.0 | 1 | — |
| Ammonium | 0.5 | 0.75 | 0.5 | 2.0 | 0.25 |
| acryloyldimethyltaurate/ | | | | | |
| VP copolymer | | | | | |
| Blue, water-soluble | 0.5 | 1.0 | 0.4 | 3.0 | 0.25 |
| dye (Blue No. 1) | | | | | |
| Carbomer | — | — | 0.2 | — | 0.1 |
| Glyceryl glucoside | 5.0 | 7.5 | 10.0 | 2.5 | 2.5 |
| Octyldodecanol | 0.5 | — | 1.0 | — | 2.0 |
| Caprylic/capric | — | — | 1.0 | — | 0.5 |
| triglyceride | | | | | |
| Myristyl myristate | — | 1.5 | 1.0 | 2.5 | — |
| Perfume | qs | qs | qs | qs | qs |
| Sea salt | — | — | 0.01 | 0.5 | — |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Glycerol | 5 | 10 | 7.5 | 15 | 7.5 |
| Tocopherol acetate | 0.5 | 0.25 | 0.5 | 0.1 | — |
| Titanium dioxide | — | 2.5 | 1.2 | 0.15 | — |
| Water | to 100 | to 100 | to 100 | to 100 | to 100 |

### O/W Emulsion

| | 31 | 32 | 33 | 34 | 35 |
|---|---|---|---|---|---|
| Polyethylene glycol(21) | 1 | — | 2.5 | 2 | 1.5 |
| stearyl ether | | | | | |
| Polyethylene glycol(2) | 1 | — | 5.5 | 3 | 7.5 |
| stearyl ether | | | | | |
| Cetearyl glucoside | — | 8 | — | — | — |
| Cyclomethicone | 12.5 | 15 | 28.0 | 25.0 | 17.5 |
| Dimethicone | 5.0 | 13.0 | 5.0 | 12.0 | 15.0 |
| Behenyl alcohol | 3 | 2 | — | 1 | — |
| Stearyl alcohol | 3 | 2 | — | 2 | — |
| Cetyl stearyl alcohol | 3 | 4 | — | — | 2 |
| Glyceryl glucoside | 0.5 | 0.75 | 11.0 | 12.5 | 6.5 |
| Octyldodecanol | 0.5 | 1.0 | 0.75 | 3.0 | 0.25 |
| Glycerol | 5 | 10 | 15 | 3 | 7.5 |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Panthenol | 0.5 | 1.0 | 0.75 | 0.25 | 0.1 |
| Perfume | qs | qs | qs | qs | qs |
| Modified starch | 0.5 | — | — | 0.15 | — |
| Water | to 100 | to 100 | to 100 | to 100 | to 100 |

### O/W Emulsion

| | 36 | 37 | 38 | 39 | 40 |
|---|---|---|---|---|---|
| Glyceryl stearate citrate | 1.0 | 0.5 | 0.1 | 0.5 | 0.3 |
| Polyethylene glycol(20) | 10.0 | 1.0 | 5 | — | — |
| cetearyl ether | | | | | |
| Triglycerine | — | — | — | — | 2.5 |
| methylglucose distearate | | | | | |
| Cyclomethicone | — | — | — | 1 | — |
| Dimethicone | 0.5 | 3.0 | 0.75 | 1.5 | 0.2 |
| Behenyl alcohol | 1 | — | 2 | 1 | 0.2 |
| Glyceryl glucoside | 3 | 5 | 10 | 15 | 5 |
| Stearyl alcohol | — | — | — | 1 | 0.2 |
| Cetyl stearyl alcohol | — | — | 1 | 1 | 0.2 |
| Tocopherol | 0.5 | 0.5 | 0.75 | 0.25 | 0.1 |
| Octyldodecanol | 0.5 | — | 0.75 | 3.0 | 0.25 |
| Panthenol | 0.5 | — | 0.75 | 0.25 | 0.1 |
| Carbomer | 0.05 | 0.35 | 0.15 | 0.1 | — |
| Perfume | qs | qs | qs | qs | qs |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Sorbitol | 10 | — | — | 5 | — |
| Butylene glycol | — | — | — | 5 | 10 |
| Propylene glycol | — | — | 10 | 5 | — |
| Glycerol | — | 7.5 | — | — | — |
| Water | to 100 | to 100 | to 100 | to 100 | to 100 |

### O/W Emulsion

| | 41 | 42 | 43 | 44 | 45 |
|---|---|---|---|---|---|
| Glyceryl stearate citrate | 1.0 | 0.75 | 0.1 | 0.5 | 0.3 |
| Polyethylene glycol(20) | 1.0 | — | 2 | 1.5 | — |
| cetearyl ether | | | | | |
| Triglycerine | — | 5.5 | — | — | 2.5 |
| methylglucose distearate | | | | | |
| Glyceryl glucoside | 5 | 10 | 15 | 20 | 7.5 |
| Cyclomethicone | 2 | 4 | 6 | 1 | 3 |
| Dimethicone | — | 0.5 | 0.75 | — | — |
| Behenyl alcohol | 1 | — | 2 | 1 | 0.2 |
| Stearyl alcohol | — | 1 | — | 1 | 0.2 |
| Cetyl stearyl alcohol | — | — | 1 | 1 | 0.2 |
| Medicinal white oil | 0.5 | 0.75 | 1.0 | 2.0 | 0.25 |
| Octyldodecanol | 0.5 | 1.0 | 0.75 | 3.0 | 0.25 |
| Panthenol | 0.5 | 1.0 | 0.75 | 0.25 | 0.1 |
| Carbomer | 0.05 | 0.1 | 0.15 | 0.1 | — |
| Perfume | qs | qs | qs | qs | qs |
| Dicaprylyl carbonate | 3 | 5 | 10 | 15 | 5 |
| Methylisothiazolinone | — | 0.10 | — | 0.06 | — |
| Benzethonium chloride | 2.0 | — | — | 0.15 | — |
| Piroctone olamine | — | — | 0.25 | 0.15 | — |
| Lauroyl ethyl arginate | — | — | — | 0.15 | 1.75 |
| Tocopherol | 0.5 | 1.0 | 0.75 | 0.25 | 0.1 |
| Caprylic/capric | 1 | 2 | 3 | 5 | 10 |
| triglyceride | | | | | |
| Modified starch | — | 2.5 | — | 0.15 | — |
| Glycerol | 3 | 5 | 8 | 12 | 10 |
| Water | to 100 | to 100 | to 100 | to 100 | to 100 |

1.-5. (canceled)

6. An active ingredient combination, wherein the combination comprises
(i) one or more preservatives selected from isothiazolinones, benzethonium chloride, piroctone olamine, and lauroyl ethyl arginate
and
(ii) one or more glucosyl glycerides.

7. The combination of claim 6, wherein (i) comprises benzethonium chloride.

8. The combination of claim 6, wherein (i) comprises piroctone olamine.

9. The combination of claim 6, wherein (i) comprises lauroyl ethyl arginate.

10. A cosmetic preparation, wherein the preparation comprises the active ingredient combination of claim 6.

11. The preparation of claim 10, wherein the preparation comprises from 0.001% to 40.00% by weight of the one or more glucosyl glycerides (ii), based on a total weight of the preparation.

12. The preparation of claim 11, wherein the preparation comprises from 0.005% to 15.00% by weight of the one or more glucosyl glycerides (ii).

13. The preparation of claim 11, wherein the preparation comprises from 0.01% to 12.00% by weight of the one or more glucosyl glycerides (ii).

14. The preparation of claim 10, wherein the preparation comprises from 0.00001% to 10% by weight of the one or more preservatives (i), based on a total weight of the preparation.

15. The preparation of claim 14, wherein the preparation comprises from 0.001% to 5% by weight of the one or more preservatives (i).

16. The preparation of claim 14, wherein the preparation comprises from 0.005% to 3% by weight of the one or more preservatives (i).

17. The preparation of claim 13, wherein the preparation comprises from 0.005% to 3% by weight of the one or more preservatives (i).

18. The preparation of claim 10, wherein the preparation further comprises from 1% to 20% by weight of one or more anionic surfactants, based on a total weight of the preparation.

19. The preparation of claim 18, wherein the one or more anionic surfactants comprise sodium lauryl ether sulfate.

20. The preparation of claim 10, wherein the preparation further comprises from 1% to 20% by weight of one or more amphoteric surfactants, based on a total weight of the preparation.

21. The preparation of claim 20, wherein the one or more amphoteric surfactants comprise cocamidopropyl betaine.

22. The preparation of claim 10, wherein the preparation further comprises one or more non-ionic surfactants.

23. The preparation of claim 22, wherein the one or more non-ionic surfactants comprise at least one of PEG-7 glyceryl cocoate and PEG-40 hydrogenated castor oil.

24. The preparation of claim 10, wherein the preparation further comprises one or more cationic polymers.

25. The preparation of claim 24, wherein the one or more cationic polymers comprise one or more polymers selected from quaternized cellulose derivatives, cationic guar derivatives, and polymeric dimethyl diallyl ammonium salts and copolymers thereof with esters and amides of acrylic acid and methacrylic acid.
